(19) United States
(12) Patent Application Publication
(10) Pub. No.: US 2020/0158742 A1
(43) Pub. Date: May 21, 2020

(54) Method of balancing a patient's canabinoid metabolite ratio

(71) Applicant: Aari Ruben, Tucson, AZ (US)

(72) Inventor: Aari Ruben, Tucson, AZ (US)

(21) Appl. No.: 16/350,441

(22) Filed: Nov. 16, 2018

(51) Int. Cl. G01N 33/94; G16H 20/10; G16H 50/20

(52) U.S. Cl. CPC G01N 33/948; G16H 50/20; G16H 20/10

(57) ABSTRACT

The invention provides a method and a structure for balancing cannabinoid metabolites within a patient. Using a stool sample, a panel of cannabinoid metabolite levels is established and compared to ideal ranges thereof. Metabolites outside of the ideal range are treated with a cannabis product or alternatively with traditional medicines.

### BACKGROUND OF THE INVENTION

This invention relates generally to medical diagnostic methods and systems and more particularly to medical considerations relative to the use of cannabis.

Recently usage of cannabis and products containing cannabis has increased with the wider acceptance of, and the benefits of, the use of cannabis for both recreational purposes as well as medical purposes. An illustration of this use is shown in U.S. Pat. No. 6,630,507, issued Oct. 7, 2003, to Hampson et al, and entitled “Cannabinoids as antioxidants and neuroprotectants”, incorporated hereinto by reference.

The medical profession is, on the whole, untrained as to the diagnosis and treatment of a physical condition that may or may not be related to cannabis use. Even further, recognition that various oils and by products of cannabis have been shown to provide significant health benefits has been overlooked by the traditional medical profession. Studies in the healthy use of cannabis are expanding this knowledge base; but it is difficult for the medical provider to keep abreast with all of the latest findings.

It is clear there is a need for a tool to assist in the diagnosis and treatment of cannabinoids within a patient.

### SUMMARY OF THE INVENTION

Within this invention, a method of balancing a cannabinoid metabolite within a patient together with a system to perform these tasks is disclosed.

Ideally a panel for cannabinoid metabolites is performed repeatedly to ascertain the effectiveness of the prescribed treatments. The ideal source used within this invention for measuring the metabolites is from a stool sample from the patient.

Using this stool sample, the diagnostic laboratory establishes a metabolite level for each of a selected group of cannabinoids. There are at least 188 different cannabinoids as well terpenes and other molecules found in cannabis (collectively referred to as cannabis metabolites). While ideally, all of the cannabinoids are analyzed, as a practical matter, a selected group is identified for testing and analysis. This selected group may be specific to the patient or may be a grouping which has been generally recognized indicators for health purposes.

These metabolites are indicative of the effectiveness of the patient's metabolism to utilize enzymes and generally show the body's health.

A variety of techniques are well known to those of ordinary skill in the art for establishing the metabolite level, including, but not limited to that described in: U.S. Pat. No. 10,006,925, issued Jun. 26, 2018, to Bitenc et al, and entitled “Methods and system for metabolite and/or lipid-based detection of colorectal cancer and/or adenomatous polyps; and U.S. Pat. No. 6,686,454, issued Feb. 3, 2004, to Yatscoff et al, entitled “Antibodies to specific regions of cyclosporine related compounds”, both of which are incorporated hereinto by reference.

The metabolite levels within the sample are used against standardization levels which establishes the ideal range for each of the metabolite levels.

When a metabolite measurement from the sample falls outside the ideal range, this fact is highlighted in a report together with a recommended cannabis product which has been found to affect the specific metabolite to adjust the sample's metabolite to fall within the ideal range.

While the preferred embodiment utilizes a cannabis product, in some embodiments, traditional medication (non-cannabis related) are used to adjust the metabolite. Those of ordinary skill in the art readily recognize a variety of such treatments, including, but not limited to those described in: U.S. Pat. No. 10,100,317, issued on Oct. 16, 2018, to Hargreaves et al, and entitled “Compositions of matter that reduce pain, shock and inflammation by blocking linoleic acid metabolites and uses thereof”; and, U.S. Pat. No. 10,047,370, issued Aug. 14, 2018, to Noguchi et al, and entitled “Tobacco enzymes for regulating content of plant metabolites, and use thereof”; both of which are incorporated hereinto by reference.

With this knowledge of the cannabis metabolite levels, and the recommended correcting cannabis product with the latest up-to-date research, the care-giver is able to communicate with a remote dispensary so that the patient is able to obtain the correcting cannabis product. In this manner, the patient is provided not only an indicator of their health, but also the proper treatment associated with the condition based on current research.

One of the main metabolite levels for this invention relates to the metabolite of cannabidiol.

While the preferred embodiment contemplates using a cannabis product as the treatment, traditional medicine may also be used. This type of treatment is often obtained from a traditional pharmacy.

The invention, together with various embodiments thereof, will be more fully explained by the accompanying drawings and the following description thereof.

### DRAWINGS IN BRIEF

FIG. 1 is a diagram illustrating the transfer of physical elements together with data transfer found in the preferred embodiment of the invention.

### DRAWINGS IN DETAIL

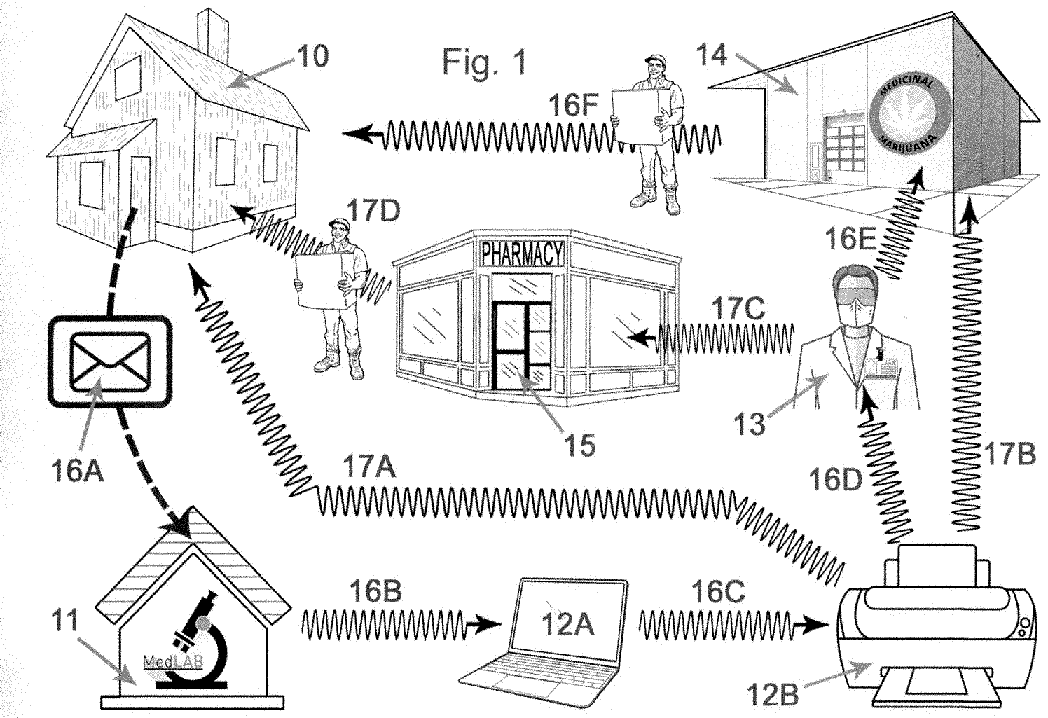

FIG. 1 is a diagram illustrating the transfer of physical elements together with data transfer found in the preferred embodiment of the invention. In this illustration the stool sample is obtained by the patient at their home 10. The invention is not so limited as the place of the collection of the sample, and includes a variety of locations obvious to those of ordinary skill in the art, including, but not limited to: a physician's office, a hospital, a clinic, and a marijuana dispensary.

The sample is communicated 16a (ideally by mail) to a laboratory 11 where the metabolites for cannabinoids are established and communicated 16B to a computer 12A where the metabolite levels are collected and correlated. Note, in some embodiments, this includes terpenes and other cannabis molecules. This information is cross referenced to current scientific research results,

In the preferred embodiment, this metabolite test includes a metabolite of Cannabidiol and in some embodiments terpenes.

In the preferred embodiment, computer 12A also collects data from other sources dealing with not only which cannabinoid metabolites are important, but also commentary from other patients together with results from scientific research.

The cannabinoid metabolite data is analyzed to determine the data's position within a range (within an ideal range or outside the ideal range as stored with computer 12A). The results of the analysis, together with recommended treatment for those metabolites outside of the ideal range, is communicated 16C to printer or other communication apparatus 12B.

In the preferred embodiment, the analysis with recommendations, is communicated 16D to care giver 13 who uses this information to prescribe a cannabis treatment 16E to marijuana dispensary 14. Using the prescription from the care giver 13, marijuana dispensary 14 delivers 16F the prescribed cannabis treatment to the patient's house 10. In an alternative embodiment, the patient retrieves the cannabis treatment from the dispensary after providing proper proof of their identity.

In some situations, a non-cannabis treated is warranted. In that case, the care-giver communicates the non-cannabis prescription 17C to a traditional pharmacy 15 where the prescription is either delivered 17D to the patient's home or is retrieved from the pharmacy 15 by the patient.

In yet another embodiment, the analysis is communicated 17B from the communication apparatus to the dispensary 14; a message is also communicated 17A to the patient's house that the dispensary 14 has the report and to retrieve the appropriate treatment.

It is clear that the present invention provides a method and structure for the proper diagnosis and treatment of cannabis conditions within the human body.

What is claimed is:

1. A method of balancing a cannabinoid metabolite within a patient comprising the steps of, repeatedly:
a) obtaining a stool sample from a patient; and,
b) via a diagnostic laboratory using the stool sample:
1) establishing a selected group of cannabinoid metabolite levels within the stool sample;
2) providing a diagnosis based upon a recommended metabolite ideal range for each metabolite with the group, if each metabolite is within the ideal range; and,
2) based upon the diagnosis, identifying a particular cannabis product which is adapted to adjust a chosen metabolite within the group of cannabinoid metabolite levels to be within an ideal range.

2. The method of balancing cannabinoid metabolites within a patient according to claim 1, further including the steps of:
a) communicating the particular cannabis product to a remote dispensary of the patient; and,
b) at the remote dispensary, obtaining valid identification of the patient and delivering to the patient the particular cannabis product.

3. The method of balancing cannabinoid metabolites within a patient according to claim 2, wherein the group of cannabinoid metabolite levels includes a metabolite of Cannabidiol.

4. the method of balancing cannabinoid metabolites within a patient according to claim 2, wherein the group of cannabinoid metabolite level includes at least one metabolite of terpenes.

5. The method of balancing cannabinoid metabolites within a patient according to claim 2, further including the step of, prescribing a non-cannabis treatment to assist in the adjustment of a selected cannabinoid metabolite level.

6. A method of balancing a cannabinoid metabolite within a patient comprising the steps of:
a) establishing a selected group of cannabinoid metabolite levels within a patient sample;
b) comparing each cannabinoid metabolite level to a historically established range;
c) providing a diagnosis, based upon a recommended metabolite ideal range for each metabolite with the group, if each metabolite is within the ideal range.

7. The method of balancing a cannabinoid metabolite within a patient according to claim 6, further comprising the steps of, based upon the diagnosis, identifying a particular cannabis product which is adapted to adjust a chosen metabolite level of the patient to be within an ideal range.

8. The method of balancing a cannabinoid metabolite within a patient according to claim 6, further including the step of, prescribing a non-cannabis treatment to assist in the adjustment of a selected cannabinoid metabolite level.

9. The method of balancing a cannabinoid metabolite within a patient according to claim 6, further including the step of obtaining a stool sample from a patient to serve as the patient sample.

10. The method of balancing a cannabinoid metabolite within a patient according to claim 9, further including:
a) providing the patient with a sample collection mechanism; and,
b) a delivery method for transmittal of the sample to a testing laboratory.

11. The method of balancing a cannabinoid metabolite within a patient according to claim 10, further including the steps of:
a) communicating the particular cannabis product to a remote dispensary; and,
b) at the remote dispensary, delivering to the patient the particular cannabis product.

12. A diagnostic laboratory for the medical treatment of a patient using cannabis adapted to:
a) receive a stool sample from a patient;
b) establish a grouping of cannabinoid metabolite levels within the stool sample; and,
c) communicate to a remote care-giver a report having,
1) the grouping of metabolite levels, and,
2) a recommended metabolite ideal range for each metabolite with the grouping.

13. The diagnostic laboratory for the medical treatment of a patient using cannabis according to claim 11, wherein the report further includes, based upon the diagnosis, identifying a particular cannabis product which is adapted to correct a chosen metabolite to be within the grouping.

14. The diagnostic laboratory for the medical treatment of a patient using cannabis according to claim 13, further including communicating the particular cannabis product to a remote dispensary of the patient

15. The diagnostic laboratory for the medical treatment of a patient using cannabis according to claim 12, delivering a non-cannabis treatment adapted to assist in the adjustment of a selected cannabinoid metabolite level to the patient.
